SEQUENCE LISTING (8 sequences)

SEQ ID NO: 1 - DNA, 21 bp, Artificial Sequence
  Description of Artificial Sequence Synthetic primer
gagcgttttgggtccaggaat

SEQ ID NO: 2 - DNA, 21 bp, Artificial Sequence
  Description of Artificial Sequence Synthetic primer
aatgcccgcaagtcaaagaaa

SEQ ID NO: 3 - DNA, 22 bp, Artificial Sequence
  Description of Artificial Sequence Synthetic primer
tcagagcccaagtacgttgaga

SEQ ID NO: 4 - DNA, 20 bp, Artificial Sequence
  Description of Artificial Sequence Synthetic primer
tgctccggttgcaagaaatt

SEQ ID NO: 5 - DNA, 21 bp, Artificial Sequence
  Description of Artificial Sequence Synthetic primer
gcggctaatgggtggaatcta

SEQ ID NO: 6 - DNA, 21 bp, Artificial Sequence
  Description of Artificial Sequence Synthetic primer
cattcttgttcagggcctcat

SEQ ID NO: 7 - DNA, 22 bp, Artificial Sequence
  Description of Artificial Sequence Synthetic primer
gaaagctcatgggaggactaga

SEQ ID NO: 8 - DNA, 22 bp, Artificial Sequence
  Description of Artificial Sequence Synthetic primer
ccacgtaactgaggaagttgac

(19) United States
(12) Patent Application Publication
(10) Pub. No.: US 2008/0188447 A1
(43) Pub. Date: Aug. 7, 2008

(54) Glucocorticoid-lowering composition

(76) Inventors: Svyatoslav Komarnytsky, Highland Park, NJ (US); Dmitry Govorko, Princeton, NJ (US); Joseph M. O'Neal, Plainsboro, NJ (US); Ilya Raskin, Manalapan, NJ (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/011,246

(22) Filed: Jan. 25, 2008

Related U.S. Application Data
(60) Provisional application No. 60/899,054, filed on Feb. 2, 2007.

(51) Int. Cl. A61K 31/56; C12N 9/00; C12N 9/02; C12N 5/06; A61P 3/00; A61P 5/00

(52) U.S. Cl. 514/169; 435/183; 435/189; 435/375

(57) ABSTRACT

This disclosure relates to a composition comprising pregnane derivatives according to Formula (I), or a salt, solvate or hydrate thereof, and to a pharmaceutically acceptable carrier or excipient. The composition may be used to lower glucocorticoid level in a human or animal. It may also be used tor modulate activity of enzymes of glucocorticoid metabolism, to modulate RNA transcription and protein level of hypothalamic, hippocampal and pituitary peptides, or for treatment or prophylaxis of human or animal diseases.

### CROSS REFERENCE TO RELATED APPLICATION

This application claims the benefit of U.S. Provisional Patent Application No. 60/899,054, filed Feb. 2, 2007, the entirety of which is hereby incorporated by reference into this application.

### FIELD OF THE INVENTION

The invention relates to compositions comprising pregnane derivatives and the use of the compositions for treatment of humans or animals having endocrinal disorders, neural-associated disorders, aging-associated disorders, chronic stress-related disorders, or certain cancers.

### BACKGROUND OF THE INVENTION

### Glucocorticoids

Glucocorticoids are stress hormones that modulate a large number of physiological actions involved in metabolic, inflammatory, cardiovascular and behavioral processes. [Wang M. The role of glucocorticoid action in the pathophysiology of the metabolic syndrome. Nutr Metab (Lond). 2005; 2(1):3]. Elevated glucocorticoid metabolism is considered a major risk factor for certain cancers like polycystic ovary syndrome [Barber T M, McCarthy M I, Wass J A H, Franks S. Obesity and polycystic ovary syndrome. Clinical Endocrinology 2006; 65(2):137-145].

Excess levels of glucocorticoids are responsible, at least partially, for risk factors associated with certain metabolic-related disorders. One of the best models for such a metabolic-related disorder is Cushing's syndrome, an endocrine disease caused by excessive levels of endogenous glucocorticoids [Orth D N. Cushing's syndrome. N Engl J. Med. 1995; 332(12):791-803]. Cushing's syndrome is characterized by multiple risk factors: persistent hypertension, insulin resistance, hyperglycemia, diabetes mellitus and heart disease [Hammer F, Stewart P M. Best Pract Res Clin Endocrinol Metab. 2006 September; 20(3):337-53].

There is a need for a single therapy that is effective for the multiple risk factors of Cushing's syndrome. There is a need, additionally, for a single therapy approach to multiple risk factor diseases generally, that would improve adherence to the single therapy drug through simplification of the drug treatment regimen and reduction of the cost of therapy overall.

Elevated glucocorticoid level has also been shown to result in low corticotrophin-releasing hormone (CRH) production in the hypothalamus, which is associated with the main neural concomitants in certain types of depression: self accusation, expectation of punishment, and crying, [De Jong J A, Roy A. Relationship of cognitive factors to CSF corticotropin-releasing hormone in depression. Am J Psychiatry. 1990; 147(3):350-2]. Moreover, heightened glucocorticoid levels impair long-term memory functions and may inhibit traumatic memory retrieval in humans [Brunner R, Schaefer D, Hess K, Parzer P, Resch F, Schwab S. Effect of high-dose cortisol on memory functions. Ann N Y Acad Sci. 2006; 1071:434-7]. Finally, glucocorticoids increase the salience of compulsive activities such as ingestion of “comfort foods” under stress conditions leading to overeating and depression [Dallman M F, Pecoraro N, Akana S F, La Fleur S E, Gomez F, Houshyar H, Bell M E, Bhatnagar S, Laugero K D, Manalo S. Chronic stress and obesity: a new view of “comfort food”. Proc Natl Acad Sci USA. 2003; 100(20):11696-701].

There is a clear need for therapeutic solutions to abnormally high glucocorticoid levels.

Glucocorticoid levels are associated with certain neural and aging disorders, as well. Older people can lose 20-25% of the cells in their hippocampus due to an elevated level of glucocorticoids. When this happens, proper feedback to the hypothalamus is lacking and a hippocampus degenerative cascade results, similar to the deterioration seen in the pancreas-insulin feedback system [Khalsa D S, Stauth S. Brain longevity. Warner Books, New York. 1999]. Smaller hippocampus, in turn, results in increased vulnerability to psychological traumas such as posttraumatic disorder [Gilbertson M W, Shenton M E, Ciszewski A, Kasai K, Lasko N B, Orr S P, Pitman R K. Smaller hippocampal volume predicts pathologic vulnerability to psychological trauma. Nat. Neurosci. 2002; 5(11):1242-7].

Additionally, an elevated level of glucocorticoids due to stress is significantly associated with known determinants of cell senescence and longevity [Epel E S, Blackburn E H, Lin J, Dhabhar F S, Adler N E, Morrow J D, Cawthon R M. Accelerated telomere shortening in response to life stress. Proc Natl Acad Sci USA. 2004 December; 101(49): 17312-5]. Overall, excess glucocorticoids may play a contributing role toward neuron death [Lee A L, Ogle W O, Sapoisky R M. Stress and depression: possible links to neuron death in the hippocampus. Bipolar Disord. 2002; 4(2):117-28]. Cushing syndrome, which is characterized by a pathologic oversecretion of glucocorticoids, is often presented with hippocampus loss [Sapolsky R M. Glucocorticoids and hippocampal atrophy in neuropsychiatric disorders. Arch Gen Psychiatry. 2000; 57(10):925-35]. Individuals with a shrunken hippocampus tend to progress more rapidly towards Alzheimer's [Petersen R C, Jack C R Jr, Xu Y C, Waring S C, O'Brien P C, Smith G E, Ivnik R J, Tangalos E G, Boeve B F, Kokmen E. Memory and MRI-based hippocampal volumes in aging and AD. Neurology. 2000; 54(3):581-7].

Bulimia nervosa, anorexia nervosa, binge eating disorder, or night eating disorder have been linked to exaggerated glucocorticoid levels in response to stress. [Koo-Loeb J H, Costello N, Light K C, Girdler S S. Women with eating disorder tendencies display altered cardiovascular, neuroendocrine, and psychosocial profiles. Psychosom Med 2000; 62: 539-48; Abell T L, Malagelada J R, Lucas A R, Brown M L, Camilleri M, Go V L, Azpiroz F, Callaway C W, Kao P C, Zinsmeister A R. Gastric electromechanical and neurohormonal function in anorexia nervosa. Gastroenterology 1987; 93: 958-65; Gluck M E, Geliebter A, Hung J, Yahav E. Cortisol, hunger, and desire to binge eat following a cold stress test in obese women with binge eating disorder. Psychosom Med. 2004; 66(6):876-81]. Among lean women, those who are high cortisol reactors ate significantly more food after a stress task [Epel E S, Lapidus R, McEwen B, Brownell K. Stress may add bite to appetite in women: a laboratory study of stress-induced cortisol and eating behavior. Psychoneuro-endocrinology 2001; 26:34-49].

What is needed is a therapy that will normalize the elevated glucocorticoid levels shown in response to stress in individuals suffering from these disorders.

However, the present pharmacotherapy for elevated glucocorticoid level has disadvantages. The known drugs are aminoglutethimide (Cytadren), ketoconazole (Nizoral), etomidate (Amidate), metyrapone (Metopirone), mitotane (Lysodren) and trilostane (Modrenal). All of these drugs fall within the category of inhibitors of steroid synthesis and are mostly used for temporary treatment of adrenal carcinoma and Cushing's syndrome. The use of these drugs as therapeutic agents in metabolic syndrome or metabolic-related disorders is limited by a number of associated, adverse side-effects.

As side effects of aminoglutethimide, are neoplasms of the adrenal cortex and thyroid follicular cells, teratogenic effects, morbilliform skin rash, drowsiness, nausea, dizziness, loss of appetite. Ketoconazole usage has been associated with adverse hepatotoxicity, anaphylaxis, hypersensitivity, nausea, vomiting, abdominal pain, dizziness, loss of appetite. Etomidate side effects are brief pain in the veins, coughing, drowsiness, hiccups, nausea, loss of appetite. Metyrapone side effects are nausea, vomiting, abdominal discomfort or pain, dizziness, sedation, allergic rash, loss of appetite. As side effects of mitotane, a very high percentage of patients have shown gastrointestinal disturbances, anorexia, nausea, vomiting, diarrhea, depression as manifested by lethargy and somnolence, dizziness or vertigo, skin toxicity. As side effects of trilostane, are darkening of skin, drowsiness or tiredness, loss of appetite, mental depression, skin rash, vomiting, diarrhea, stomach pain. This product was withdrawn from the U.S. market in April 1994.

What is needed is a new therapeutic that is without the abundant side effects of these drugs.

### Pregnanes

Pregnanes are a class of naturally occurring saturated steroid substances widely distributed in the plant kingdom and in some marine organisms. They are structurally related to cardiac glycosides, however they express a wide range of biological activity including antitumor, cytotoxic, platelet pro-aggregating, antifungal, acetylcholine esterase inhibiting, and anti-osteoporotic properties [Dal Piaz F, De Leo M, Braca A, De Simone F, Morelli 1, De Tommasi N. Electrospray ionization mass spectrometry for identification and structural characterization of pregnane glycosides. Rapid Commun Mass Spectrom. 2005; 19(8):1041-52].

Several plant pregnane glycosides possess appetite-suppressing properties such as compositions described in U.S. Pat. No. 6,376,657 to Van Heerden et al. (genus Hoodia), U.S. Pat. No. 7,008,648 to Corley et al. (genera Stapelia and Orbea); U.S. Patent Application Publication No. 20060084638 to Raskin et al. (genus Asclepias). It has been suggested that Hoodia pregnanes alter ATP levels in the hypothalamus, yet no specific molecular target or pathway has been identified [MacLean D B, Luo L G. Increased ATP content/production in the hypothalamus may be a signal for energy-sensing of satiety: studies of the anorectic mechanism of a plant steroidal glycoside. Brain Res. 2004; 1020(1-2):1-11].

Certain pregnane glycosides from Asclepias were identified earlier [Warashina T, Noro T. Steroidal glycosides from the aerial part of Asclepias incarnata. Phytochemistry 2000; 53(4):485-498] and [Roy M C, Chang F R, Huang H C, Chiang M Y, Wu Y C. Cytotoxic principles from the formosan milkweed, Asclepias curassayica. J Nat Prod. 2005; 68(10):1494-9]. No biological or therapeutic activity for these compounds was disclosed.

### SUMMARY OF THE INVENTION

One aspect of the present invention encompasses a therapeutic method comprising administering to a subject a composition comprising a pregnane derivative according to Formula (I) or a salt, solvate or hydrate thereof, wherein the composition lowers the circulating glucocorticoid level. In an alternative aspect of the invention, the therapeutic method contemplates administering the same composition to a subject who has a higher than normal glucocorticoid level. It is contemplated that the therapeutic method is for a human or veterinary therapy.

In one embodiment, the therapy is for an endocrinal or a metabolic-related disorder that is a hormonal imbalance or a defect in central energy regulation or central energy expenditure. In one contemplated embodiment, the therapy is for Cushing's syndrome.

In still another embodiment the therapy is a treatment for cancer. Polycystic ovary syndrome is one contemplated cancer.

In yet another embodiment, the therapy is a treatment for a neural-associated disorder. In certain embodiments, the neural-associated disorder is selected from the group consisting of faulty synaptic plasticity, learning and memory impairment, faulty memory retrieval, faulty neural cell regeneration, depression, and posttraumatic disorder.

In another embodiment the therapy is a treatment for aging-associated disorders. Some of the contemplated disorders are associated with oxidative stress, low telomerase activity and short telomere length.

In yet another embodiment, the therapy is for a chronic stress disorder and the composition alleviates stress. In still another embodiment the therapy is for bulimia nervosa, anorexia nervosa, binge eating disorder, night-eating disorder and comfort food cravings.

In still another embodiment, what is contemplated is a method for modulating the activity of a factor or enzyme of glucocorticoid metabolism comprising contacting the factor or enzyme with a pregnane derivative according to Formula (I) or a salt, solvate or hydrate thereof. The contacting is in vivo or in vitro. The factor or enzyme is selected from the group consisting of StAR, CYP11A1, HSD3B2, CYP17, CYP21A2, CYP11B1, CYP11A2, CYP11B2, HSD11B1, and CYP3A4.

The invention further encompasses a method for modulating RNA transcription of a hypothalamic or pituitary peptide comprising contacting the hypothalamic, hippocampal or pituitary cell with a pregnane derivative according to Formula (I) or a salt, solvate or hydrate thereof. The contacting is in vivo or in vitro. The hypothalamic, hippocampal or pituitary peptide is selected from the group consisting of ACTH, CRH, POMC, CART, NPY, AgRP, AVP, OXT, α-MSH and β-MSH.

Also contemplated is a method for modulating the level of a hypothalamic, hippocampal or pituitary peptide comprising contacting the hypothalamic, hippocampal or pituitary cell with a pregnane derivative according to Formula (I) or a salt, solvate or hydrate thereof. The contacting is in vivo or in vitro. The hypothalamic, hippocampal or pituitary peptide is selected from the group consisting of ACTH, CRH, POMC, CART, NPY, AgRP AVP, OXT, α-MSH and β-MSH.

### DETAILED DESCRIPTION OF THE INVENTION

### Definitions

The scientific literature has adopted a number of terms. For consistency and clarity, the following definitions will be used throughout this patent document.

“Glucocorticoid-lowering” is intended herein to encompass a statistically significant and detectable or measurable reduction in circulating glucocorticoid levels in a human or animal body (over a time period of at least about 12 hours).

As used herein, by “modulating” is intended to mean inhibiting, activating, modifying, regulating, or controlling at a statistically significant level.

“Factors and enzymes of the glucocorticoid metabolism” is intended herein to encompass factors that regulate or enzymes that perform at least one enzymatic step of glucocorticoid synthesis or modification in the human or animal body.

“Hypothalamic, hippocampal and pituitary peptides” is intended herein to encompass peptides that are expressed, spliced, translated, modified post-translationally, produced, secreted, or released by neuroendocrine cells of the hypothalamus, hippocampus, or pituitary of a human or animal and the level of the peptides is modulated, directly or indirectly, by glucocorticoids.

“Neural associated disorders” is intended herein to encompass disorders involving synaptic plasticity, learning and memory impairment, decreased memory retrieval, neural cell regeneration, depression, and posttraumatic disorder.

“Age-associated disorders” is intended herein to encompass disorders associated with higher oxidative stress, lower telomerase activity, and shorter telomere length, which are known determinants of senescence and longevity.

“Chronic stress disorders” is intended herein to encompass, in addition to chronic stress itself, excess “comfort” food intake, bulimia nervosa, anorexia nervosa, binge eating disorder, or night eating disorder.

“Comfort food” is intended herein to encompass palatable foods having caloric value which have sensory qualities producing a sense of comfort.

### Structural Information

The present invention encompasses the glucocorticoid-lowering composition comprising pregnane derivatives according to Formula (I):

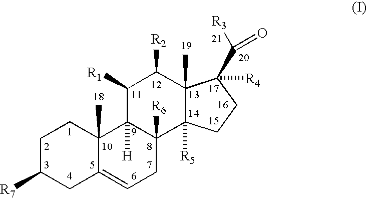

wherein:
R₁ (if present) is a C₁-C₁₈ moiety;
R₂ is hydroxyl, or a C₁-C₁₈ moiety;
R₃ is hydroxyl, or a C₁-C₁₈ moiety;
R₄ is hydrogen, hydroxyl, or a C₁-C₁₈ moiety;
R₅ is hydrogen, hydroxyl, or a C₁-C₁₈ moiety;
R₆ is hydrogen, hydroxyl, or a C₁-C₁₈ moiety;
R₇ is hydrogen, hydroxyl, keto, or a C₁-C₁₈ moiety, or a saccharide moiety;
and the dotted line represents an optional double bond between C₅ and C₆.

“C₁-C₁₈ moiety” as used herein includes from one to 18 carbon atoms. Typical examples include alkyl, alkylene, heteroalkyl, alkenyl, acyl, and aryl groups as defined herein. In some embodiments, an individual substituent may be described by more than one of the aforementioned terms.

“Alkyl” as used herein includes straight chain and branched hydrocarbon groups containing up to 18 carbon atoms, for example, one to ten, and one to eight carbon atoms.

“Alkylene” as used herein includes alkyl groups (as defined) further including one or more substituents.

“Heteroalkyl” as used herein includes alkyl groups further containing a heteroatom such as O, P, S, or N.

“Alkenyl” as used herein includes alkyl groups further containing one or more carbon-carbon double bonds.

“Acyl” as used herein includes a substituent having the chemical formula —CO—R₈ wherein R₈ is a moiety, as defined above, but containing between one and 17 carbon atoms. In preferred embodiments, R₈ is selected from the group consisting of aryl and alkylene, particularly alkylenearyl (i.e., an alkylene group having an aryl substituent). Representative acyl groups include formyl, acetyl, propionyl, butyryl, benzoyl, toluoyl, phenylacetyl, tigloyl, cinnamoyl, and nicotinoyl. In some embodiments, R₂ is preferably an acyl group, particularly benzoyl, tigloyl, cinnamoyl, and nicotinoyl. In some embodiments, the acyl group includes an aryl group containing a heteroatom, as described below.

“Aryl” as used herein includes a monocyclic or polycyclic aromatic group, preferably a monocyclic or bicyclic aromatic group, e.g., phenyl or naphthyl. An “aryl” group can be unsubstituted or substituted, for example, with one or more, and in particular one to three, halo, alkyl, alkoxy, alkoxyalkyl, haloalkyl, nitro, and/or cyano substituents. The aryl group may also contain one or more heteroatoms such as O, P, S, or N.

“Saccharide moiety” as used herein includes a pentose, hexose, heptose, or octose sugar, analog, or derivative thereof, including, but not limited to, deoxy sugars, dideoxy sugars, amino sugars, and sugar acids. The term includes disaccharides, oligosaccharides, and polysaccharides, which are comprised of two or more saccharides that are joined by a glycosidic linkage.

The pregnane derivatives according to Formula (I) are steroidal compounds. In one embodiment, the steroidal compounds are pregnane glycosides (i.e., R₇ is a saccharide moiety). In another embodiment, the steroidal compounds are pregnane aglycones (i.e., R₇ is hydrogen).

The pregnane derivatives according to Formula (I) belong to a class of naturally occurring C₂₁ steroid compounds designated by IUPAC as pregnanes. These compounds are widely distributed in living things such as microorganisms to higher animals.

The methods for preparing a botanical extract from Asclepias plants enriched with pregnane derivatives according to Formula (I), as well as for isolation and identification of individual pregnane derivatives have been disclosed in U.S. Patent Application Publication No. 2006/0084638, incorporated herein in its entirety.

### Glucocorticoid-Lowering Activity

The invention relates to a glucocorticoid-lowering composition comprising pregnane derivatives according to Formula (I). Because of this glucocorticoid-lowering property, what is contemplated is the use of these compositions to modulate activity of enzymes and factors of glucocorticoid metabolism (e.g., CYP11A1 and the like).

Steroid synthesis of glucocorticoids starts with the four-ring structure of cholesterol and involves factors (StAR and the like) and enzymes of glucocorticoid synthesis: CYP11A1 (cholesterol to prenenolone), HSD2B2 (prenenolone to progesterone), CYP17 (progesterone to 17-hydroxyprogesterone), CYP21A2 (17-hydroxyprogesterone to 11-deoxycortisol), CYP11B1 (11-deoxycortisol to cortisol). Progesterone is also a substrate for mineralocorticoid synthesis through CYP11A2 to yield deoxycorticosterone and CYP11B2 to yield aldosterone [Miller, W L. Molecular biology of steroid hormone synthesis. Endocr Rev 1988; 9:295]. At a tissue-specific level (especially in the liver and adipose tissue), the availability of cortisol to bind to the glucoccorticoid receptors is regulated by a bidirectional enzyme HSD11B1, which regenerates active glucocorticoids [Tomlinson J W, Stewart P M. Mechanisms of disease: Selective inhibition of 11beta-hydroxysteroid dehydrogenase type 1 as a novel treatment for the metabolic-related syndrome. Nat Clin Pract Endocrinol Metab. 2005; 1(2):92-9]. Additionally, CYP3A4 is a major liver enzyme that is involved in metabolism and degradation of glucocorticoids [Nelson D R, Koymans L, Kamataki T, Stegeman J J, Feyereisen R, Waxman D J, Waterman M R, Gotoh O, Coon M J, Estabrook R W, Gunsalus I C, Nebert D W. P450 superfamily: update on new sequences, gene mapping, accession numbers and nomenclature. Pharmacogenetics 1996; 6:1-42].

The C₁₁ position is critical for hormonal-related activity of glucocorticoids. For example, the active human glucocorticoid cortisone is produced from inactive 11-deoxycortisone by hydroxylation (addition of a single hydroxyl moiety) to C₁₁ position. Furthermore, the active human glucocorticoid cortisone is easily modified into its inactive form cortisone by dehydrogenation (removal of a single hydrogen moiety) from the C₁₁ position.

The present invention also relates to use of a glucocorticoid-lowering composition comprising pregnane derivatives according to Formula (I) to modulate RNA transcription and level of hypothalamic, hippocampal, and pituitary peptides (e.g., POMC and the like).

The present invention also relates to use of the glucocorticoid-lowering composition comprising pregnane derivatives according to Formula (I) for treatment or prophylaxis of human or animal diseases such as endocrinal disorders, metabolic-related disorders, neural-associated disorders, aging-associated disorders, chronic stress and stress-associating disorders, and certain cancers.

The pharmaceutical composition comprising pregnane derivatives according to Formula (I) has glucocorticoid-lowering activity as demonstrated herein in EXAMPLE 1. Thus, a glucocorticoid-lowering composition comprising at least one of the pregnane derivatives according to Formula (I) finds application as an anti-glucocorticoid agent, and hence a therapeutic or prophylactic drug for endocrinal disorders, neural-associated disorders, aging-associated disorders, chronic stress and stress-associating disorders, and certain cancers.

Additionally, it is contemplated that the glucocorticoid-lowering composition will be administered therapeutically to subjects with bulimia nervosa, anorexia nervosa, binge eating disorder, or night eating disorder to alleviate the exaggerated cortisol response to stress that such subjects demonstrate.

The invention encompasses the use of natural or synthetic compositions comprising pregnane derivatives according to Formula (I) in accordance with the invention, for example, in solution, mixture, powder, dried preparation and the like to effectively suppress, inhibit, reduce or otherwise curtail glucocorticoid level in a human or animal.

In preparing the above therapeutic glucocorticoid-lowering composition comprising pregnane derivatives according to Formula (I), other pharmaceutically acceptable components may be mixed appropriately in a conventional manner. While not intending to restrict the choice of such components, an exemplary list includes an excipient, disintegrator, lubricant, binder, antioxidant, coloring agent, flocculation inhibitor, absorption promoter, solubilizer, stabilizer, etc.

The dosage form for the above glucocorticoid-lowering composition comprising pregnane derivatives according to Formula (I) is not particularly restricted but can be freely selected from among liquid solution, suspension, emulsion, tablet, pill, capsule, sustained release formulation, powder, suppository, oral formulation, liposome, microparticle, microcapsule, sterile isotonic aqueous buffer solution, sterile isotonic aqueous buffer solution comprising anesthetic, etc.

The above glucocorticoid-lowering composition comprising pregnane derivatives according to Formula (I) can be administered transdermally, intramuscularly, intraperitoneally, intravenously, subcutaneously, intranasally, by inhalation, intralesionally, endothelially, topically, orally, by oral mucosa, rectally, by intestinal mucosa, etc.

The amount and dosage regimen of the above glucocorticoid-lowering composition comprising pregnane derivatives according to Formula (I) is based on various factors relevant to the purpose of administration, for example age, sex, body weight, hormone levels, or other needs for a particular treatment or prophylaxis. A typical dosage is about 0.01 mg/kg body weight/day to about 200 mg/kg body weight/day, preferably about 5 mg/kg body weight/day to about 50 mg/kg body weight/day.

### Decreased Glucocorticoid State Results In Appetite Suppression

The glucocorticoid-lowering composition comprising pregnane derivatives according to Formula (I) in accordance with the invention possess appetite-suppressive properties. Appetite can be restored with glucocorticoid supplementation as disclosed in EXAMPLE 2. For example, injection of low doses of glucocorticoid sufficient to saturate only 50% of the glucocorticoid receptors in the rodent model successfully restored appetite in these animals to the level of 80% of that of the control animals.

Because the glucocorticoid-lowering composition comprising pregnane derivatives according to Formula (I) reduced food intake indirectly by lowering plasma glucocorticoid level, the inventive concept encompasses the use of the composition to treat or prevent compulsory eating disorders by eliminating food cravings, particularly of “comfort foods”.

### Modulation of Glucocorticoid Metabolism

The glucocorticoid-lowering composition comprising certain pregnane derivatives according to the Formula (I) possesses appetite-suppressive properties that can be restored with glucocorticoid supplementation, as disclosed in EXAMPLE 3. Administration of low doses of various glucocorticoids sufficient to saturate only 50% of the glucocorticoid receptors in the rodent model successfully restored appetite in these animals to the level of up to 80% of that of control animals.

When intermediate metabolites of the steroid biosynthesis pathway were administered, some degree of restoration of appetite was observed. These data point to the conclusion that the glucocorticoid-lowering composition comprising certain pregnane derivatives according to Formula (I) modulates at least CYP11A1 or HSD2B2 enzyme.

Because the glucocorticoid-lowering composition comprising pregnane derivatives according to Formula (I) reduced food intake indirectly by lowering plasma glucocorticoid level, the inventive concept encompasses the use of the composition to treat or prevent compulsory eating disorders by eliminating food cravings, particularly of “comfort foods”.

The following examples illustrate the present invention in further detail, it being understood that these examples do not limit the scope of the invention.

### EXAMPLE 1

### Plasma Glucocorticoid-Lowering Effect

Male Wistar rats (Charles River Laboratories, Wilmington, Mass.) weighing about 300 g (5 individuals per group) were put on regular Purina rodent chow diet for feeding and tap water ad libitum from day 1 to day 3 of the experiment. Animals were housed individually in cages in a temperature-controlled environment (22° C.) with 12 h light/12 h dark cycle; lights off at 1400. On days 1 and 2, a botanical extract from a plant Asclepias incarnata enriched with pregnane derivatives according to Formula (I) was administered orally in doses of 100 mg/kg/day in 1 ml of corn oil (Sigma #C8267, St. Louis, Mo.). As a negative control (vehicle), animals were administered orally with 1 ml of corn oil alone. On day 3, the blood was drawn from each animal and the plasma was separated at about 1300-1400 (beginning of the dark period, “sunset”) and 0100-0200 (beginning of the light period, “sunrise”). The amount of corticosterone, a major glucocorticoid in rodents, was measured by ELISA according to the manufacturer's instructions (Assay Designs #900-097, Ann Arbor, Mich.).

The results are presented in Table 1. The data in Table 1 show that the botanical extract enriched with pregnane derivatives according to Formula (I) lowers total corticosterone in rodent plasma. The botanical extract enriched with pregnane derivatives according to Formula (I) causes a marked decrease in the corticosterone level at the “sunset” time point, which is expected to peak at this time, as can be seen in control animals. Corticosterone is a major glucocorticoid in rodents (homologous to cortisol in humans).

TABLE 1

Plasma corticosterone (glucocorticoid), ng/ml
Time:

| Group | sunrise | sunset |
|---|---|---|
| Control | 4.55 ± 2.01 | 22.17 ± 5.92 |
| Treated | 5.45 ± 2.21 | 9.57 ± 1.31** |

**Significant at p < 0.01

### EXAMPLE 2

### Lower Glucocorticoid Levels are Responsible for Modulation of Appetite

The male Wistar rats weighing about 250 g (3 groups, 6 individuals per group) were kept as described in the Example 1 from day 1 to day 5 of the experiment. On days 1 through 4, a botanical extract from a plant Asclepias incarnata enriched pregnane derivatives according to Formula (I) was administered orally in doses of 100 mg/kg body weight/day in 1 ml of corn oil. As a negative control (vehicle), animals were administered orally with 1 ml of corn oil alone. A pair fed group of animals received the average amount of food consumed by the animals treated with the pregnane derivatives according to Formula (I). Daily food intake was recorded on day 4, and body weight gain was calculated over the period of 4 days of the experiment. On day 5 of the experiment the blood was drawn from each animal and the plasma was separated. The concentration of glucose and insulin in plasma was measured by Amplex Red Glucose/Glucose Oxidase Assay Kit (Invitrogen #A-22189, Carlsbad, Calif.) and Linco Rat/Mouse Insulin ELISA Kit (Linco Research #EZRMI-13K, St. Charles, Mich.), respectively. The results are presented in Table 2.

TABLE 2

| | Daily food | Body weight | Plasma | Plasma |
|---|---|---|---|---|
| Group | intake, g | gain, g | glucose, mM | insulin, pM |
| Control | 21.8 ± 1.1 | 31.9 ± 1.4 | 6.5 ± 0.3 | 224.3 ± 36.1 |
| Treated | 14.7 ± 0.7** | 25.2 ± 3.1* | 6.0 ± 0.3 | 193.5 ± 27.2 |
| Pair fed | same as for | 20.9 ± 2.2* | 4.6 ± 0.4* | 123.7 ± 13.5* |
| | Treated group | | | |

Significant at **p < 0.01; *p < 0.05

The data in Table 2 show that, in the control group, where the animals ate ad libitum, their plasma glucose is at the normal value of 6.5 mM. In contrast, animals treated with the formula (I) compounds reduced their food intake. The animals in this group had a slightly lower plasma glucose of 6 mM and a similar effect on insulin levels.

### EXAMPLE 3

### Low Glucocorticoid State Induced by Modulation of the Enzymes of Steroid Synthesis

Male Wistar rats weighing about 250 g (2 groups designated as group C, control; and E, experimental treatment; 24 individuals per group) were kept as described in the Example 1 from day 1 to day 6 of the experiment. On days 1 through 4, a botanical extract from a plant Asclepias incarnata enriched with pregnane derivatives according to Formula (I) was administered orally in doses of 100 mg/kg body weight/day in 1 ml of corn oil (Sigma #C8267). As a negative control (vehicle), animals were administered orally with 1 ml of corn oil alone. On day 4 of the experiment both groups of animals C and E were further subdivided into 6 groups containing 4 animals each, designated C, control; PREG, pregnenolone; PROG, progesterone; DEOX, deoxycorticosterone; CORT, corticosterone; and DHEA, dehydroepiandrosterone. On day 4 and day 5, animals from these subgroups received a subcutaneous injection of 5 mg/kg body weight/day of the respective steroid compound in 5% DMSO and corn oil; total volume of 0.1 ml. The control animals in group C subgroup C and group E subgroup C received a subcutaneous injection of vehicle (5% DMSO in corn oil) only. On days 4 and 6 food intake was measured. The results are presented in Table 3.

TABLE 3

Daily food intake, g

| Group | Inj. | Abbr. | Day 4 | Day 6 |
|---|---|---|---|---|
| Control | No | C-C | 24.0 +− 1.6 | 25.7 +− 0.8 |
| Control | Yes | C-PREG | 23.9 +− 1.5 | 29.4 +− 0.9* |
| Control | Yes | C-PROG | 22.5 +− 0.9 | 30.0 +− 1.3* |
| Control | Yes | C-DEOX | 24.2 +− 1.5 | 25.4 +− 2.1 |
| Control | Yes | C-CORT | 25.1 +− 0.5 | 25.2 +− 1.1 |
| Control | Yes | C-DHEA | 23.6 +− 1.3 | 24.2 +− 1.5 |
| Treated | No | E-C | 16.0 +− 1.2* | 17.3 +− 1.9 |
| Treated | Yes | E-PREG | 18.9 +− 0.7* | 23.7 +− 1.6 |
| Treated | Yes | E-PROG | 17.5 +− 1.1* | 24.9 +− 1.2 |
| Treated | Yes | E-DEOX | 16.1 +− 1.4* | 25.2 +− 0.9 |
| Treated | Yes | E-CORT | 16.9 +− 1.3* | 23.2 +− 1.3 |
| Treated | Yes | E-DHEA | 18.3 +− 1.6* | 19.5 +− 0.8* |

Inj, glucocorticoid injection on days 4 and 5; Abbr., group abbreviation.
*Significant at p < 0.05 as compared to control

The data from Table 3 show that the botanical extract enriched with pregnane derivatives according to the Formula (I) suppresses appetite in treated animals (compare food intake in groups C-C and E-C on day 4). However, two daily subcutaneous injections of various glucocorticoids as listed were sufficient to partially restore appetite in all subgroups, suggesting that synthesis of glucocorticoids in the treated animals is blocked at the level of the first enzyme (CYP11A1, responsible for synthesis of pregnenolone) or the second enzyme (HSD3B2, responsible for synthesis of progesterone). No such effect was observed in the control group receiving a vehicle injection (subgroup E-C). The smallest possible corticosterone dose to saturate about 50% of glucocorticoid receptors in rat for a day was used in this experiment according to published data [Karten Y J, Nair S M, van Essen L, Sibug R, Joels M. Long-term exposure to high corticosterone levels attenuates serotonin responses in rat hippocampal CA1 neurons. Proc Natl Acad Sci USA. 1999; 96(23):13456-61]; this dose had no direct effect on food intake alone as evident from the control group that received corticosterone injection (subgroup C-CORT).

### EXAMPLE 4

### RNA Transcript Modulation

The animals were treated as described in Example 1. Fresh brains and adrenal glands were collected on day 3 of the experiment.

Total RNA was extracted using RNeasy Mini Kit (Qiagen, Germantown, Md.) following the manufacturer instructions. RNA was quantified spectrophotometrically by absorption measurements at 260 nm and 280 nm using the Nanoprop system (Nanoprop Technologies, Wilmington, Delware). Quality of RNA was assessed by separation gel-electrophoresis. RNA was then treated with Dnasel (Invitrogen, Carlsbad, Calif.) following manufacturer guidelines, to remove any traces of DNA contamination. The cDNAs were synthesized using 2 μg of RNA for each sample using Stratascript™ reverse transcriptase (Stratagene, La Jolla, Calif.), following the manufacturers' protocol. The synthesized cDNAs were diluted 4-fold. 5 μl of each sample were used for PCR reactions of 25 μl final volume. The other components of the PCR reactions were 0.5 μl of 6 μM gene specific primers (IDT, Coralville, Iowa), 12.5 μl of Brilliant SYBR green PCR master mix (2×) (Stratagene, La Jolla, Calif.) containing green jump-start Taq ready mix. ROX (Stratagene, La Jolla, Calif.) was used as an reference dye. The following primers were designed using Primer Express 2.0 software (Applied Biosystem, Foster City, Calif.): cyclophilin (ref NM_{—}017101) forward primer 5′-GAG CGT TTT GGG TCC AGG AAT-3′ (SEQ ID NO: 1), reverse primer: 5′-AAT GCC CGC AAG TCA AAG AAA-3′ (SEQ ID NO: 2); CRH (ref NM_{—}031019) forward primer TCA GAG CCC AAG TAC GTT GAG A (SEQ ID NO: 3), reverse primer TGC TCC GGT TGC AAG AAA TT (SEQ ID NO: 4); HSD3B2 (ref NM_{—}000198), forward primer GCG GCT AAT GGG TGG AAT CTA (SEQ ID NO: 5), reverse primer CAT TCT TGT TCA GGG CCT CAT (SEQ ID NO: 6); HSD11B1 (ref NM_{—}005525) forward primer GAA AGC TCA TGG GAG GAC TAG A (SEQ ID NO: 7), reverse primer CCA CGT AAC TGA GGA AGT TGA C (SEQ ID NO: 8). Intron-spanning primers were used. Real-time PCR amplifications were performed on MX3000p system (Stratagene, La Jolla, Calif.) using 1 cycle at 50° C. for 2 min, 1 cycle of 95° C. for 10 min, followed by 40 cycles of 15 sec at 95° C. and 1 min at 60° C. The dissociation curve was completed with one cycle of 1 min at 95° C., 30 sec of 55° C. and 30 sec of 95° C. NRT (non-RT control) and NTC (no template control) were included in each experiment as quality control steps. RNA expressions for POMC, normalized with respect to the expression of housekeeping cyclophilin gene, were analyzed using the ΔΔCt method [Winer et al., Development and validation of real-time quantitative reverse transcriptase-polymerase chain reaction for monitoring gene expression in cardiac myocytes in vitro. Analytical Biochemistry 1999; 270:41-9]. The ΔΔCt values obtained from these analyses directly reflect the relative mRNA quantities for the specific gene in response to a particular treatment as compared to a calibrator. The hypothalamus tissue from untreated animals served as a calibrator sample in this study. The value of the POMC gene expression in the calibrator sample was assigned a value of 1.0. A value more than 1.0 indicates transcriptional up-regulation (increase in gene expression) as compared to the calibrator. Amplification of specific transcripts was further confirmed by obtaining melting curve profiles. All samples were run in duplicate.

TABLE 4

| | CRH | HSD3B2 | HSD11B1 |
|---|---|---|---|
| Group | (fold expression difference relative to the calibrator gene [actin]) | | |
| Control | 1.0 | 1.0 | 1.0 |
| Treated | 3.23 | −2.43 | −2.11 |

The glucocorticoid-lowering composition comprising certain pregnane derivatives according to the Formula (I) modulates expression of the CRH gene in the hypothalamus and HSD3B2 and HSD11B1 genes in the adrenal gland, as shown in Table 4.

1. A therapeutic method comprising administering to a subject who has a higher than normal glucocorticoid level a composition comprising a pregnane derivative according to Formula (I) or a salt, solvate or hydrate thereof, wherein said composition lowers the circulating glucocorticoid level.

2. The method of claim 1 wherein the therapy is for an endocrinal or metabolic-related disorder that is a hormonal imbalance or a defect in central energy regulation or central energy expenditure.

3. The method of claim 2 wherein the therapy is for Cushing's syndrome.

4. The method of claim 1 wherein the therapy is a treatment for a neural-associated disorder.

5. The method of claim 1 wherein the therapy is for a chronic stress disorder and the composition alleviates stress.

6. The method of claim 1 wherein the therapeutic method is for a human or veterinary therapy.

7. A method of prophylaxis for a defect in central energy regulation or expenditure comprising administering to a subject a composition comprising a pregnane derivative according to Formula (I) or a salt, solvate or hydrate thereof, wherein said composition modifies the metabolism to decrease energy expenditure.

8. A method for modulating the activity of a factor or enzyme of glucocorticoid metabolism comprising contacting the factor or enzyme with a pregnane derivative according to Formula (I) or a salt, solvate or hydrate thereof.

9. The method of claim 8 wherein the contacting is in vivo.

10. The method of claim 8 wherein the contacting is in vitro.

11. The method of claim 8 wherein the factor or enzyme is CYP11A1 or HSD3B2.

12. A method for modulating RNA transcription of a hypothalamic, hippocampal or pituitary peptide comprising contacting a hypothalamic, hippocampal or pituitary cell with a pregnane derivative according to Formula (I) or a salt, solvate or hydrate thereof.

13. The method of claim 12 wherein the contacting is in Vivo.

14. The method of claim 12 wherein the contacting is in vitro.

15. The method of claim 12 wherein the peptide is CRH.

16. A method for modulating the level of a hypothalamic, hippocampal or pituitary peptide comprising contacting a hypothalamic, hippocampal or pituitary cell with a pregnane derivative according to Formula (I) or a salt, solvate or hydrate thereof.

17. The method of claim 16 wherein the contacting is in vivo.

18. The method of claim 16 wherein the contacting is in vitro.

19. The method of claim 16 wherein the peptide is CRH.
